(19) United States
(12) Patent Application Publication
(10) Pub. No.: US 2011/0066227 A1
(43) Pub. Date: Mar. 17, 2011

(54) Stent with Dynamic Drug Reservoirs

(75) Inventors: Michael P. Meyer, Richfield, MN (US); Timothy S. Girton, Edina, MN (US); Dennis Boismier, Shorewood, MN (US); Daniel Gregorich, St. Louis Park, MN (US); Kim Robertson, Forest Lake, MN (US); Liza Davis, St. Michael, MN (US); Cory Hitzman, St. Paul, MN (US); Samuel Robaina, Santa Rosa, CA (US); Doug Thesingh, Minneapolis, MN (US)

(73) Assignee: BOSTON SCIENTIFIC SCIMED, INC., Maple Grove, MN (US)

(21) Appl. No.: 12/561,781

(22) Filed: Sep. 17, 2009

(51) Int. Cl. A61F 2/06

(52) U.S. Cl. 623/1.42; 623/1.15

(57) ABSTRACT

A stent having one or more beneficial agents which enhance the healing effect of the stent. The agent is positioned within a reservoir. The reservoir dynamically releases the agent when the stent is expanded. The reservoir makes use of various changes in stent shape during expansion to assure proper release of the agent. Agent release can be modulated at various points along the stent and with various rates of release. The reservoirs can also be cooperatively combined with features that enhance stent flexibility, structural integrity, and which prevent recoil.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use and more particularly to intravascular stents that include a plurality of cavities formed on one or more surfaces of the stent and are coated with drugs

2. Description of the Related Art

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable). Stents may be implanted to prevent restenosis following angioplasty in the vascular system.

Implantable medical devices are often used for delivery of a beneficial agent, such as a drug, to an organ or tissue in the body at a controlled delivery rate over an extended period of time. Proper release of those agents however, has proved to be difficult. The surface of the stent cannot merely be coated with one or more agents because such a coating is exposed to water, other compounds, or conditions in the body which degrade the drugs. Placing the agent within a shallow hole drilled into the surface of the stent doesn't adequately protect the agent from the body's conditions. Placing the agent within a deep hole excessively inhibits adequate release of the agent. Thus there is a need for a stent having an agent within a reservoir that is both adequately protected from the body's conditions until properly released, yet which allows for proper agent release.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

At least one embodiment is directed to an expandable stent constructed of a plurality of interconnected stent members. The stent comprises one or more reservoirs of beneficial agent positioned on a stent member. When expanded the agent is less securely retained within the reservoir than when unexpanded. The reservoir is positioned at a focal point in the member which becomes more deformed by expansion into the expanded state than adjacent portions of that member. The deformation causes the change in retention of the agent within the reservoir.

At least one embodiment is directed to a stent having generally linear adjacent struts joined by turns. Circumferentially and longitudinally offset turns can be interconnected by connectors. The reservoir can be located along the outside or inside of the turns and can be deformed by tension and compression. The reservoir can be tapered to be narrower closer to the turn and wider farther away from the turn. The turn can also have a width greater than that of the adjoining strut members. The focal point can be formed at the apex of a concave or convex bend.

The reservoir can be constructed in one shape selected from the list consisting of: channel shaped, teardrop shaped, square shaped, circular shaped, oval shaped, and any combination thereof. In some embodiments the shape of the reservoir is selected from any polygonal shape. In some embodiments the shape of the reservoir is any shape having one or more negative inflection points (e.g. I-beam shaped, C-shaped, S-shaped, etc.). The reservoir can be a hole cut into the solid material of a stent member, the walls of the reservoir descending into the solid member material according to curved, tapered, triangular, square, trapezoidal, and any combination thereof and can extend partially or entirely through the stent member material all the way to an inflation balloon the stent is crimped upon.

The beneficial agent can be a material which in the expanded state is broken by the deformation and releases fragments or can be a flexible material which becomes highly stretched by the deformation and releases through increased surface area contact. The agent can also be covered by a capping material which prevents contact with the agent until the cap is broken by the deformation in the expanded state. The agent can be positioned above a layer of material, in the expanded state the material breaks and pushes agent out of the reservoir.

The stent members include strut columns interconnected by connector members, a portion of the connector defining an opening within which a reservoir is positioned, the opening becoming deformed by the expansion of the stent. The connectors can span between turns that face away from each other and which pull the connector apart when expanded or that span between turns that face towards each other and which compress the connector. The compression and/tension in the connector deform the reservoir and release the agent.

This and other aspects of the invention are described in more detail in the accompanying description and drawings.

### BRIEF DESCRIPTION OF THE SEVERAL VIEWS OF THE DRAWINGS

The invention is best understood from the following detailed description when read in connection with accompanying drawings, in which:

FIG. 1 shows a side view of an example PRIOR ART stent.

FIG. 2A shows a stent member of an unexpanded stent with an agent filled reservoir hole.

FIG. 2B shows a stent member of an expanded stent with an agent filled reservoir hole.

FIG. 2C shows a stent member of an expanded stent with an agent filled reservoir hole.

FIG. 3A shows a stent member of an unexpanded stent with agent filled reservoirs at narrow sections of the member.

FIG. 3B shows a stent member of an unexpanded stent with agent filled reservoirs at narrow sections of the member and in holes in struts.

FIG. 3C shows a turn of a stent with reservoirs having tapered perimeters.

FIG. 4A shows a turn of a stent with a square reservoir perimeter.

FIG. 4B shows a turn of a stent with a channel shaped reservoir.

FIG. 4C shows a turn of a stent with a cap over the reservoir.

FIG. 4D shows a turn of a stent with a channel reservoir covered by a cap.

FIG. 5A shows a cross section of an unfilled reservoir.

FIG. 5B shows a cross section of an unfilled reservoir on a crimped stent.

FIG. 5C shows a cross section of a filled reservoir.

FIG. 5D shows a cross section of a releasing reservoir.

FIG. 5E shows a PRIOR ART stent.

FIG. 5F shows a stent with acutely angled turns.

FIG. 6A shows a channel filled with agent in an unexpanded stent.

FIG. 6B shows a channel filled with agent in an expanded stent.

FIG. 6C shows a turn in the unexpanded state with undeformed reservoirs.

FIG. 6D shows a turn in the expanded state with deformed reservoirs.

FIG. 6E shows a turn in the unexpanded state with an aperture opened channel.

FIG. 6F shows a turn in the expanded state with an aperture opened channel.

FIG. 6G shows a perspective view of a turn in the unexpanded state with an aperture opened channel.

FIG. 7A shows a turn in the unexpanded state with a channel reservoir and a small-hole reservoir.

FIG. 7B shows a turn with grooved reservoirs.

FIG. 7C shows a balloon pushed reservoir.

FIG. 8A shows an unexpanded stent column with closed pore reservoirs.

FIG. 8B shows an expanded stent column with open pore reservoirs.

FIG. 9A shows a section of a stent in the unexpanded state with a reservoir cell.

FIG. 9B shows a section of a stent in the unexpanded state with a reservoir cell.

FIG. 10A shows a stent in the unexpanded state with reservoir cells.

FIG. 10B shows a stent in the expanded state with reservoir cells.

FIG. 10C shows a section of a stent in the unexpanded state with reservoir cells.

FIG. 10D shows a section of a stent in the expanded state with reservoir cells.

FIGS. 11A-11F shows connectors having reservoirs within them.

FIG. 12A shows a reservoir cell bound by a curved truss.

FIG. 12B shows a reservoir cell bound by a straightened curved truss.

FIGS. 13A-13H shows reservoirs on overlapping stent members.

FIGS. 14A-14C shows agent reservoirs within ratcheting cell ribs.

### DETAILED DESCRIPTION OF THE INVENTION

The numerous different embodiments of the invention will next be illustrated with reference to the figures wherein the same numbers indicate similar elements in all figures. Such figures are intended to be illustrative rather than limiting and are included herewith to facilitate the explanation of the apparatus of the present invention.

For the purposes of this disclosure, like reference numerals in the figures shall refer to like features unless otherwise indicated.

Depicted in the figures are various aspects of the invention. Elements depicted in one figure may be combined with, or substituted for, elements depicted in another figure as desired.

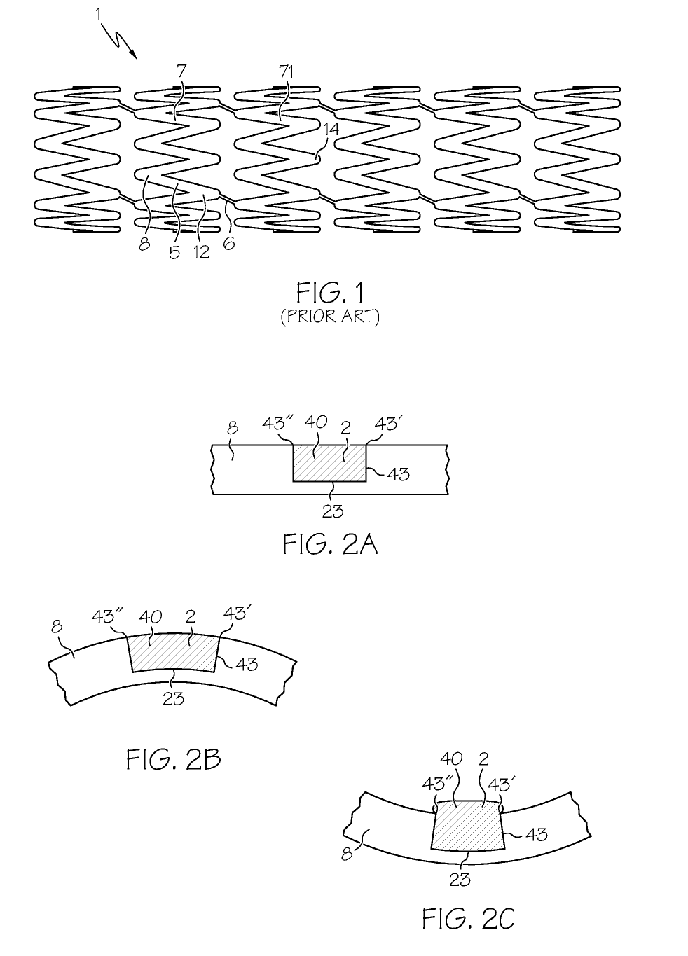

Referring now to FIG. 1, there is shown a PRIOR ART unexpanded stent (1). The stent comprises a number of stent members (8) including but not limited to: struts (5), connectors (6), and turns (12) which together define a fluid lumen (14). For purposes of this application, a turn (12) is a non-linear, often curved stent member (8) bridging at least some of the space between two adjacent struts (5) in the same strut column (7). A connector (6) is a stent member (8) which bridges at least some of the space between two stent members (8) in adjacent strut columns (7i). Connectors (6) can adjoin turns (12) and/or struts (5).

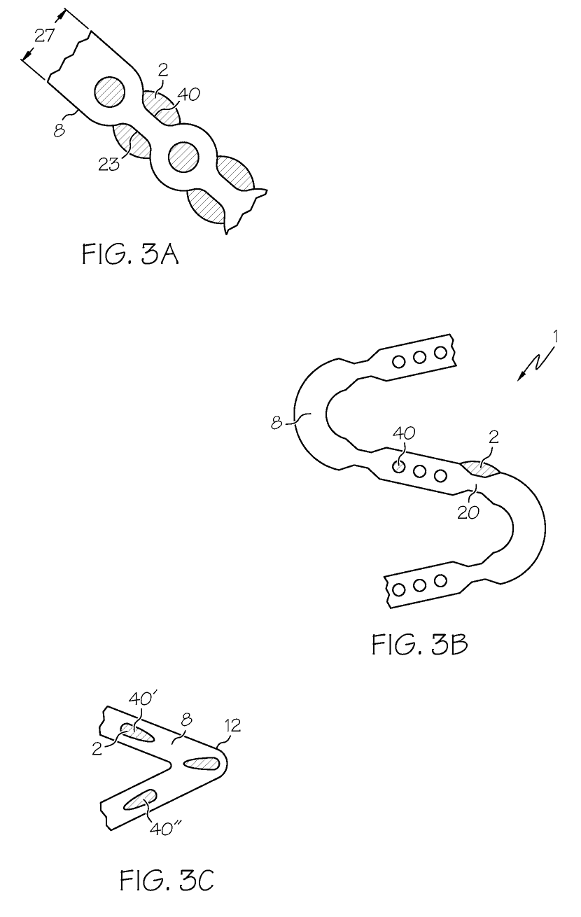

Embodiments of the present invention however, may include various structural features (struts, connectors, etc.) of the type of stent shown in PRIOR ART FIG. 1, as well as others. Regardless of the particular geometry or design of the stent, embodiments of the present invention will include the additional structural feature of at least one drug retaining reservoir (40) in one or more stent members (8), examples of which are depicted in FIGS. 2A-2C. A unique aspect of the of the present invention, is that at least some of the reservoirs (40) are configured on the stent, such that in a first state of the stent a reservoir retains the therapeutic agent (2), but when the stent is expanded to a second state, the shape of the reservoir (40) is changed, so as to initiate the release of the therapeutic agent (2) contained therein.

As shown in the various figures, a stent member (8), such as a strut, connector, etc, includes at least one reservoir (40). A reservoir (40) is a hole, channel, incision, or other void in the solid material of a stent member or some other form of storage site within which a suitable quantity of agent (2) can be contained.

The agent (2) can be at least one or various types of therapeutic agents including but not limited to: at least one restenosis inhibiting agent that comprises drug, polymer and bio-engineered materials or any combination thereof. In addition, the agent can be a therapeutic agent such as at least one drug, or at least one other pharmaceutical product such as non-genetic agents, genetic agents, cellular material, etc. Some examples of suitable non-genetic therapeutic agents include but are not limited to: at least one anti-thrombogenic agent such as heparin, heparin derivatives, vascular cell growth promoters, growth factor inhibitors, etc.; Paclitaxel, and similar compounds. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate. It will be appreciated that other types of agents or substances, well known to those skilled in the art, can be applied within the reservoir or to other portions of the stent as well. In at least one embodiment, the agent is a material which when moved by the deformation of the reservoir, breaks and releases agent.

Referring now to FIGS. 2B and 2C there are shown different embodiments in which one or more reservoirs (40) are located along focal points (23) of the stent. For the purposes of this invention at least one focal point (23) exists on a stent member that undergoes a change in area of 15% or more when the stent transitions from its unexpanded state to it expanded state. The localized strain can be relative to a radial or circumferential perspective and includes bending, twisting, flexing, stretching, compressing, and similar motions in one or more portions of one or more stent members. A focal point (23) is a position on a stent member where the localized strain induces a greater degree of deformation than in adjacent areas and where the deformation causes sufficient movement to shear away, squeeze out, push off, or otherwise dislodge material engaged to the member that would otherwise remain engaged for a period of time. Depending on the nature of the localized strain, a member can have one or more focal points with similar or greater degrees of deformation. The positioning of a reservoir (40) on a focal point (23) facilitates release of the therapeutic agent (2) by utilizing this dislodging effect. Focal points (23) can be positions on struts, connectors, turns or other portions of the stent, or combinations thereof.

When the agent reservoir (40) undergoes the deformation, the surface tension, chemical bonds, or other mechanism of restraint keeping the agent within the reservoir is overcome and the agent (2) is released at least in part from the stent. FIG. 2B illustrates at least one embodiment of a focal point (23) where the convex bending of the member (8) pulls apart the portion of the agent relative to its configuration in unexpanded state (FIG. 2A) resulting in a second degree of retention lower than the first degree of retention. FIG. 2B shows that the increase in reservoir volume accompanying the stent's expansion, no longer retains the agent in place, and therefore initiates its release.

In at least one embodiment, it is the tops (43′, 43″) of the sidewalls (43) of the reservoir (40) that wedge the agent (2) in place and when the tops of the sidewalls move apart, the agent (2) is released.

FIG. 2C illustrates at least one embodiment of a focal point (23) where the concave bending of the member (8) compresses the agent (2) within the reservoir (40). This compression can squeeze the agent out of the reservoir (40) or can simply break the polymer matrix or cap holding the agent (2) in place, resulting in a second degree of retention which is lower than the first degree of retention present in the unexpanded state (of FIG. 2A).

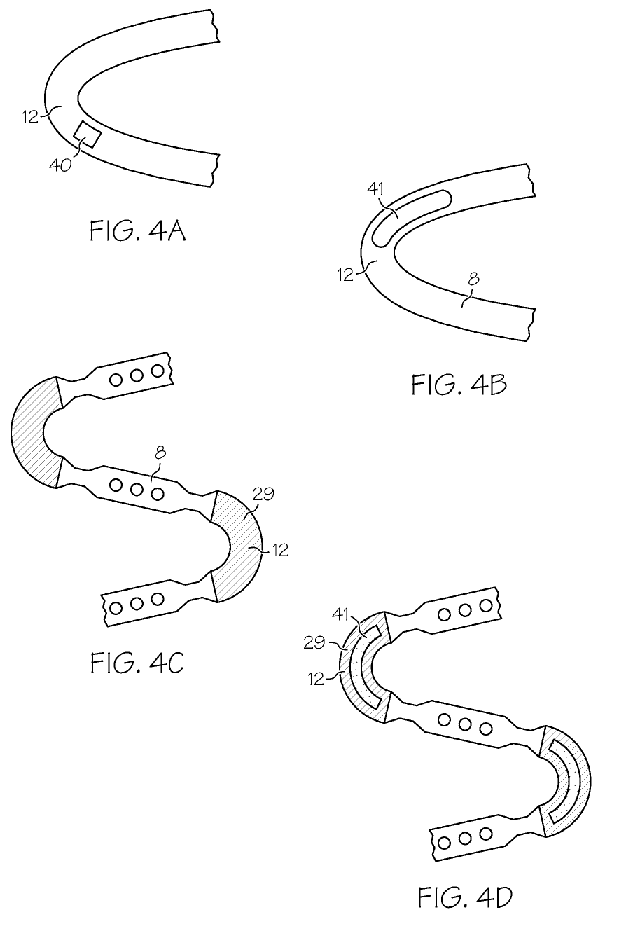

In at least one embodiment, the reservoir itself contributes the presence or location of a focal point. For example, the presence of a particularly shaped hole in a member results in a section of the member which is thinner than adjacent sections and which is more likely to bend in response to bending moments imposed on the entire member than other sections of that member. The bending then occurs precisely at the reservoir's position which in turn deforms the reservoir and releases the agent or at least increases the exposed surface area of the agent in contact with the body vessel. FIG. 3A illustrates at least one embodiment of a stent member (8) which has a generally uniform width (27). At portions of the member where there are reservoirs (40) however, the width of the member is significantly lessened. The reduced width causes the location of the agent (2) to be the actual focal point (23) and the deformation of the member upon transitioning into the expanded state facilitates the agent's (2) release.

Referring now to FIG. 3B there is shown at least one embodiment in which the stent comprises one or more ductile hinges (20) such as those described in U.S. Pat. No. 7,160,321, of which its entire content is incorporated herein by reference. The ductile hinge (20) is a specifically located narrow member section which facilitates the bending of the member (8). The reservoir (40) is positioned over the ductile hinge (20). As the hinge (20) undergoes a significant deformation during stent expansion, the hinge disrupts the stability of the agent (2) which causes release. As shown in FIG. 3B, reservoirs (40) can be positioned along other portions of the stent as well.

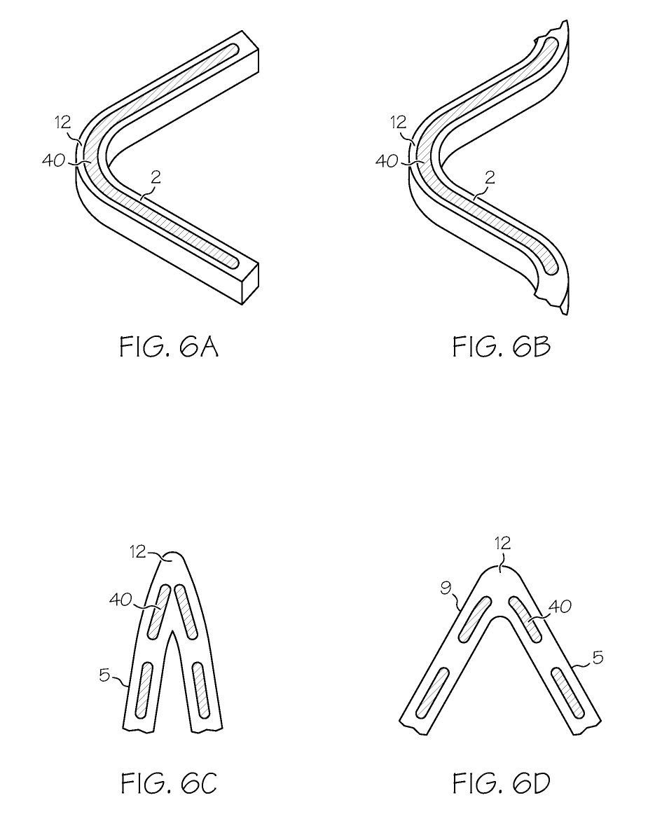

FIG. 3C also shows a deforming reservoir (40ii) having a tapered aperture which is wider at or near the turn and narrows with distance from the turn. This structure will release large amounts of agent at or near the turn and less farther from the turn. As can be seen by comparing FIGS. 6C and 6D, the deformation is more acute at the turn and near turn area and as a result for two similarly sized reservoirs, the one closer to the turn will release more than the one farther from the turn. FIG. 6C shows an unexpanded strut-turn junction having reservoirs (40) of roughly the same size. FIG. 6D shows that as the stent expands, the reservoirs (40) closer to the turns (12) undergo significant deformation with respect to their eventual shape and proximity to the edges (9) of the member while the reservoirs (40) farther from the turn are less deformed.

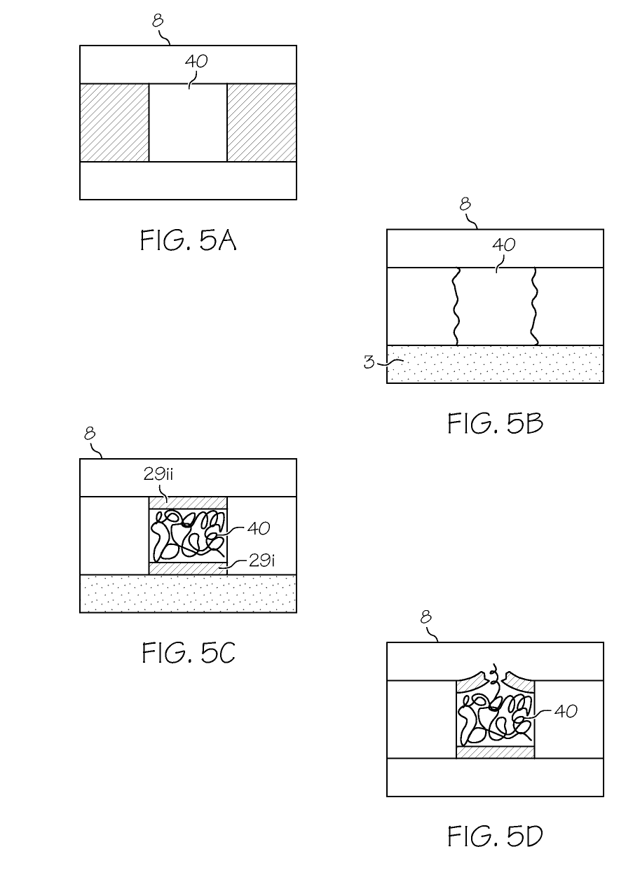

Referring now to FIG. 4C there is shown an embodiment in which the agent is covered with a cap (29). A cap (29) is a material which is brittle and/or less water soluble when compared to the substance of the agent. In at least one embodiment, the cap is dissolvable or bioabsorbable at a rate different than that of the agent. In the unexpanded state, the cap (29) covers the agent and protects the agent from premature release or degradation. When the stent is expanded, the cap (29) is ruptured. The rupture can be a result of the cap (29) breaking or the disruption of surface tension between the cap (29) and the stent member (8). In one embodiment, the agent and the cap (29) are positioned along those portions of the turn (12) and adjoining member which undergo the greatest degree of deformation. This design is of particular use with highly flexible agents which do not otherwise release well in the absence of deformation or some other manner of motion.

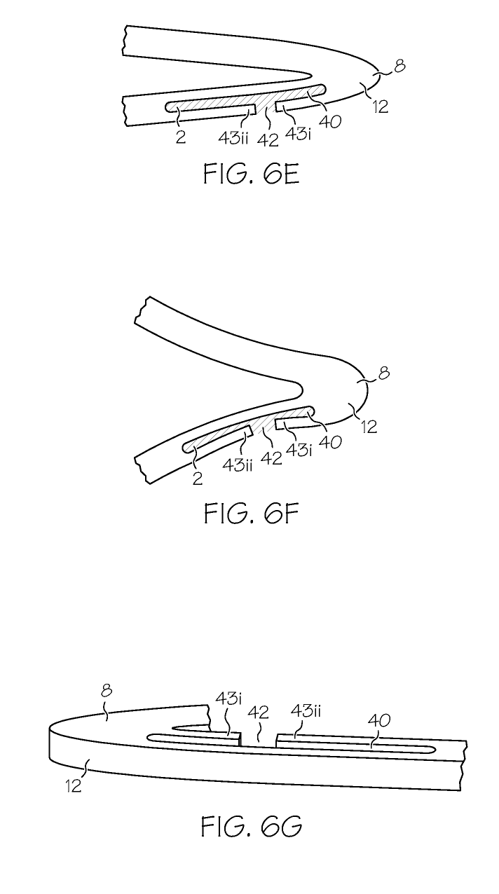

Reservoirs located at or near turns can be in a number of shapes. FIG. 4A shows a reservoir (40) in a turn (12) having a square shape. FIG. 4B shows a reservoir (40) in the form of a channel (41). A channel (41) is an elongated hole in a stent member (8) designed to run generally along the length of at least a portion of the member (8). Using channels at or near the turn can have a number of advantages. A channel gives far more flexibility to a member than a shorter hole would. In addition, a channel (41) can reduce the surface area of the turn (12) that comes into contact with the body vessel. This can be useful for protocols that call for a uniform amount of surface area contact along at least a portion of the stent and can counteract at least in part the fact that a given area of the stent containing a turn has more solid surface area than the same given area which only includes struts. In addition, because of the large surface to surface contact that occurs by turns, a channel that provides a large reservoir area can result in significant agent contact with the body vessel. Finally because of the length relative to width of a channel, the channel combined with the deformation present at a turn efficiently breaks the agent and thus has a high release rate. FIG. 4D shows a turn (12) having a channel (41) shaped reservoir with a cap (29) coating over at least some of the agent. FIG. 7A shows am embodiment in which a small reservoir (40) is positioned near the turn (12) while a larger channel (41) extends along at least a portion of the length of a strut (5).

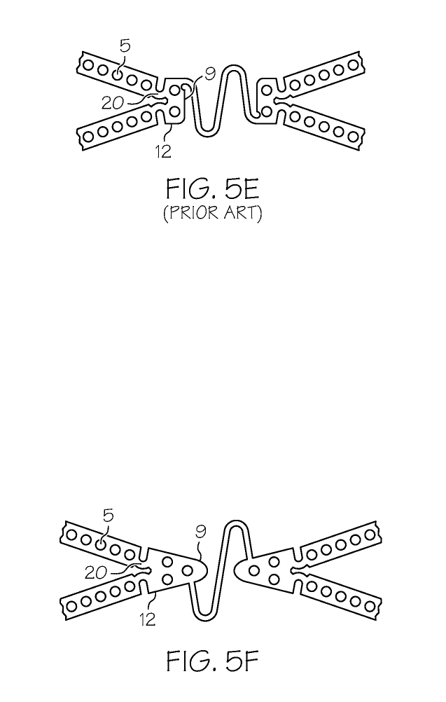

Referring now to FIGS. 5A through 5D there is shown a process for constructing and deploying a stent having a capped agent reservoir. In at least one embodiment the cap is a layer of material that is fluid impermeable or with reduced fluid permeability. In at least one embodiment the reservoir is sealed by the cap in the unexpanded state. FIG. 5A shows a close up cross section of a stent member (8) which is cut and polished and comprises a reservoir (40) for storing an agent. In FIG. 5B the stent is then crimped over a balloon (3). In FIG. 5C the reservoir (40) of the balloon crimped stent has a lower layer of cap (29i) material placed within the reservoir (40), then a layer of one or more agents (2) is placed over the lower cap layer (29i), then an upper cap layer (29ii) is placed over the agent (2) sealing the agent (2) within a cap sandwich. In FIG. 5D it is shown that when the stent is deformed, the upper layer (29ii) breaks exposing the agent (2) to the body vessel and the lower layer (29i) breaks which pushes the agent (2) out past the cracked upper agent (29ii) and against the body vessel.

Contemplated embodiments also include reservoirs with only an upper cap layer or only a lower cap. In addition, the cap breaking may be a result of compressive forces (such as those illustrated in FIG. 2C) or tensile forces (such as those illustrated in FIG. 2B). In addition, the cap can be retained to either the reservoir or the agent by an adhesive, by innate adhesive properties of the cap material or agent, or by being frictionally engaged in the volume of the reservoir.

Referring now to FIG. 5E there is shown a portion of a PRIOR ART stent disclosed in U.S. Pat. No. 7,160,321 (which is incorporated by reference in its entirety) in which the stent comprises ductile hinge (20) linking a blunt turn (12) with a strut (5). The turns (12) of this stent have a blunt leading edge (9) which causes a disruption in laminar blood flow. In contrast, the portion of the stent shown in FIG. 5F, has a turn (12) whose leading edge (9) is less blunt (so as to form an acute angle) which minimizes flow disruptions.

Referring now to FIGS. 6A and 6B there is shown an embodiment in which the deformation at a turn (12) and near turn area causes the reservoir (40) to be stretched in length and thus facilitate the release of the agent (2). In FIG. 6A, when the stent is in the unexpanded state, the agent (2) is positioned within reservoir (40) that has a defined volume. FIG. 6B shows that as the stent expands, portions of the reservoir (40) become pulled away from each other. This pulling action allows body fluid to penetrate inside of the reservoir (40) and reduces intra-agent binding forces, all of which facilitate the release of the agent (2). In at least one embodiment, the agent (2) is a flexible material which stretches in response to the stent expansion.

FIGS. 6E and 6F illustrate another embodiment in which the deformation at a turn (12) facilitates the release of an agent (2). In FIG. 6E there is shown member (8) of an unexpanded stent having a channel with an aperture (42) filled with agent. The channel may be open to the face of the member (as shown in FIG. 6G) or it may be a hollow lumen only exposed to the outside of the member through the aperture (42). The aperture (42) is bound by side walls (43i, 43ii) which define the opening in the member's face. FIG. 6F shows that as the stent expands, at least some of the side walls (43i, 43ii) bend inward compressing the channel. This compression forces the agent (2) out of the member (8) and against the body vessel. In the embodiment of FIG. 6G however, walls (43i, 43ii) bend outward upon deployment, thereby opening the aperture (42) to release/expose the agent (2).

Referring now to FIG. 7B there is shown an embodiment in which the turn region (12) in the unexpanded state comprises one or more cut grooves (40) along the outer side (9) of the member. These grooves (40) are configured to close up upon stent expansion. As the grooves (40) close up, agent positioned within the grooves (40) are squeezed out and efficiently released into the body vessel. FIG. 7C shows a cross sectional view of a stent member having a tapered reservoir (40). This taper extends from the outer face of the member (9) to an expansion balloon (3) the stent is crimped over. As the balloon (3) is inflated, the wider volume not filled by solid stent material allows the growing balloon (3) to move into the reservoir (40). The introduction of the balloon (3) displaces agent (2) contained within the reservoir (40) to initiate drug release.

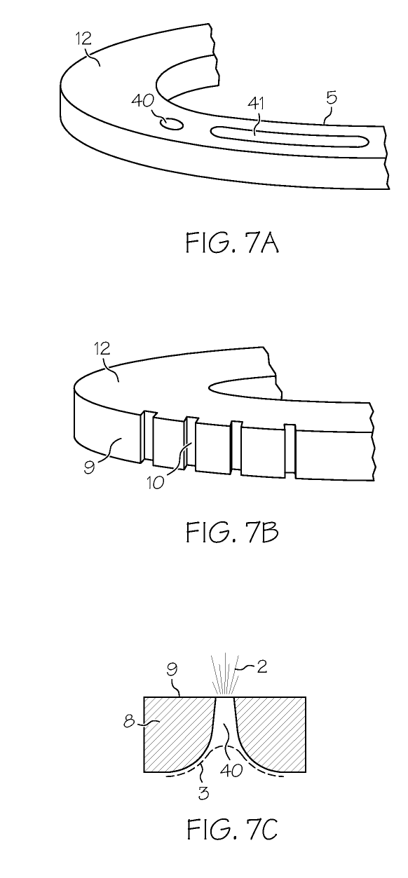

Referring now to FIG. 8A there is shown an embodiment in which an unexpanded stent comprises a number of pores (40) positioned at or near the turns (12) of a strut column (7). These pores (40) are unconnected portions of stent material within which agent (2) can be positioned. Because when the walls of the pores (40) are compacted together the agent avoids exposure to body vessel environments. As a result, agents (2) which are highly susceptible to degradation can be safely stored and can transit through body safely. As shown in FIG. 8B, upon stent expansion as the column (7) is at least partially straightened out, the pores (40) flex open and immediately expose or release the agent (2) to the body vessel.

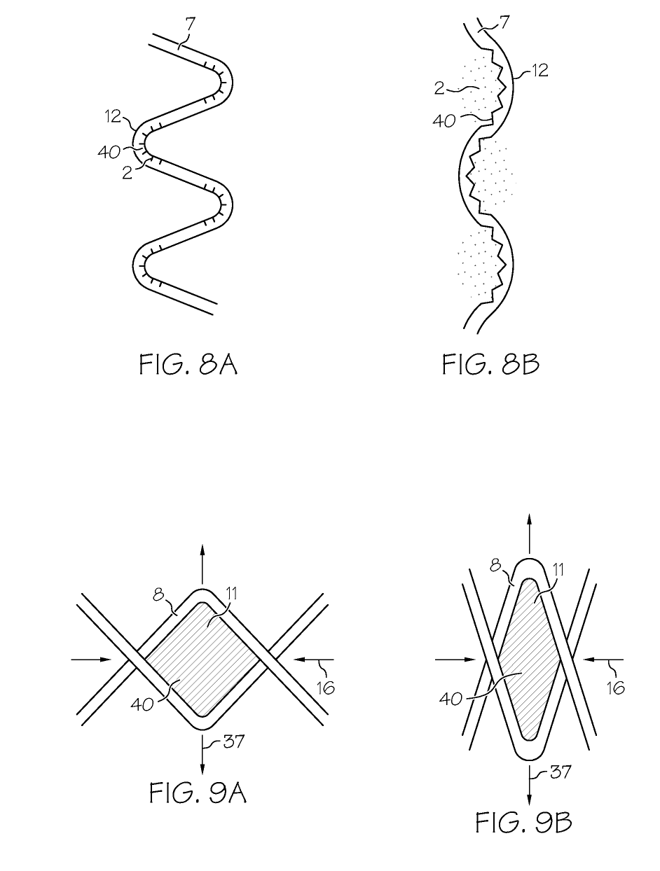

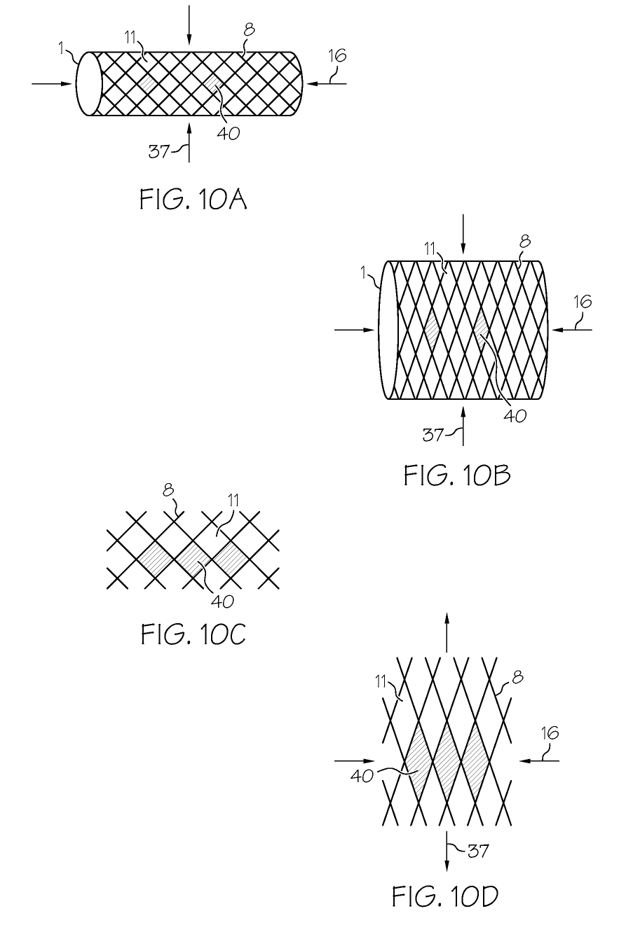

Referring now to FIGS. 10A and 10B there are shown stent (1) in which cells (11) define the walls of an agent reservoir (40). The cells (11) are openings in the wall of the stent (1) and are defined by stent members (8) such as struts, turns, and connectors. FIGS. 10C and 9A show close up views of the cells (11) of an unexpanded stent including cells (11) with in which are agent reservoirs. FIGS. 10D and 9B show that when the stent is expanded, the cell (11) becomes deformed. In one embodiment the cell (11) deformation is characterized by tension along the circumferential directions (37), and compression along the longitudinal directions (16). These deformations can facilitate release through stretching and increasing exposure of the agent to the body vessel.

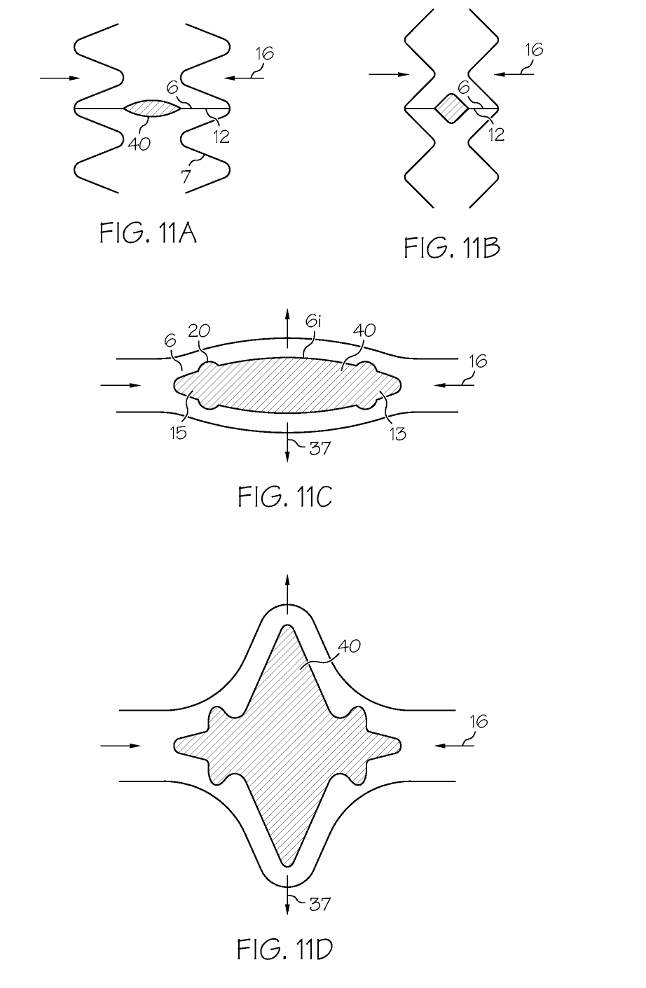

Referring now to FIG. 11A there is shown an agent reservoir (40) positioned within a connector (6) spanning between two strut columns (7) of an unexpanded stent. As FIG. 11B shows, as stent expands, the columns (7) at least partially straighten out. The connector (6) which spans between turns (12) that point away from each other tends to be deformed by an outward pulling force imposed by the moving turns (12). This pulling force deforms the reservoir (40) causing release through breaking or stretching action.

FIG. 11C shows a connector (6) reservoir (40) of the unexpanded stent. The reservoir is surrounded by a perimeter of connector material (6i). There is at least one ductile hinge (20) at or near the proximal (15) or distal (13) end of the reservoir perimeter (6i). This ductile hinge (20) has a width which is narrower than the adjacent portion of the reservoir perimeter (6i). As FIG. 11D shows, when the stent assumes its expanded state, the reservoir (40) perimeter (6i) expands circumferentially (37) and narrows longitudinally (16). The ductile hinge (20) allows the reservoir (40) to more easily flex and appropriately deform. The agent or cap in contrast cannot respond to the deformation as efficiently and as a result the reservoir releases agent.

FIG. 11E illustrates an embodiment in an unexpanded stent has a connector (6) with a reservoir (40) spanning between two turns (12) that are pointed towards each other. In this embodiment as shown in FIG. 11F when the stent expands, the proximal (15) and distal (13) ends of the perimeter (6i) diverge and the central portions of the perimeter (6i) converge, effecting the opposite deformation pattern of that shown in FIG. 11B.

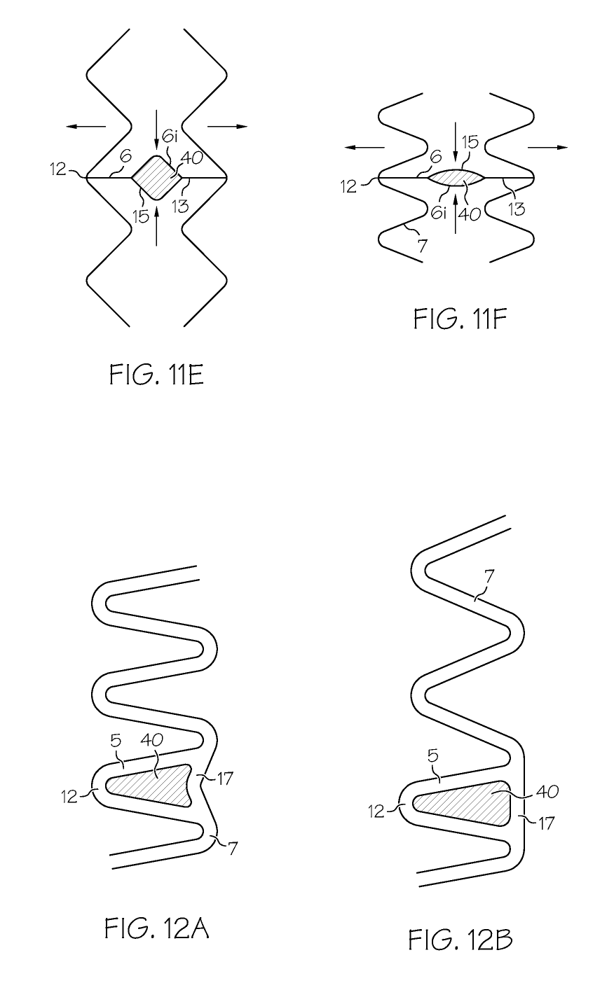

Referring now to FIG. 12A there is shown an embodiment in which the reservoir (40) is bound by pair of adjacent struts (5), a turn (12) and a truss (17) extending between the strut pair (5) on the opposite side of the column (7) as the turn (12). In the unexpanded state, the truss (17) is curved with similarly concavity to the turn (12). As FIG. 12B shows, when the stent is expanded, the truss (17) becomes straighter which deforms at least a portion of the reservoir (40) to similarly straighten as well. This deformation facilitates agent release.

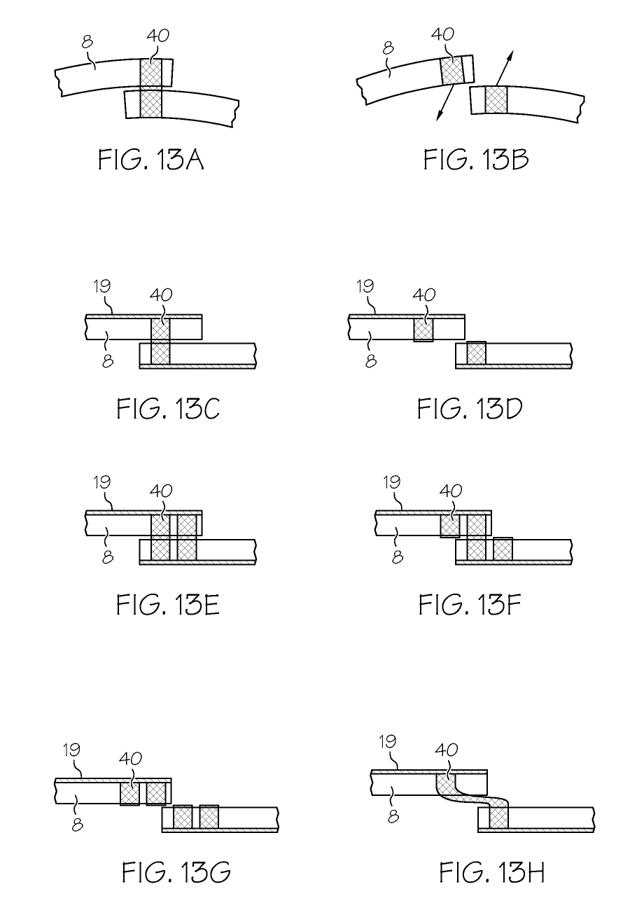

Referring now to FIG. 13A there is shown a reservoir (40) positioned along a band wrapped around two overlapping stent members (8) in an unexpanded stent. As FIG. 13B shows, when the stent expands, the overlapping members (8) are pulled away form each other. This pulling sunders the reservoir (40). In one embodiment the reservoir has a cap over it and the pulling tears open the cap freeing the agent for release. In another embodiment, the pulling breaks a solid mass of agent causing release and increased exposed surface area. FIG. 13C illustrates an unexpanded stent member (8) similar to that of FIG. 13A but which also includes a sealing cover (19). This cover (19) when positioned over a swath of the agent inhibits agent release. As FIG. 13D shows, when the members (8) are pulled away from each other, the agent is moved out from under the cover (19) and is capable and better agent release.

FIGS. 13E, 13F and 13G illustrate the members of 13C which are capable of finely regulating the levels of agent release. In FIG. 13E the overlapping members (8) have more than one agent reservoir (40). When the members (8) are pulled they can be pulled to a lesser extent exposing one reservoir (40) or only a portion of a reservoir (40), or they can be pulled to a greater extent exposing more or all of the reservoirs (40) and thus facilitating enhanced levels of agent release. In embodiments where the members (8) are capable of more than one-way overlapping movements, this mechanism can facilitate activating and deactivating release or modulating the release with degrees of fine motor control. FIG. 13H illustrates an embodiment in which the moved overlapping members (8) have an expandable agent (40) on them, whereby the expansion pulls the agent out from under the cover (19) and stretches the agent (40) increasing its surface area and release rate.

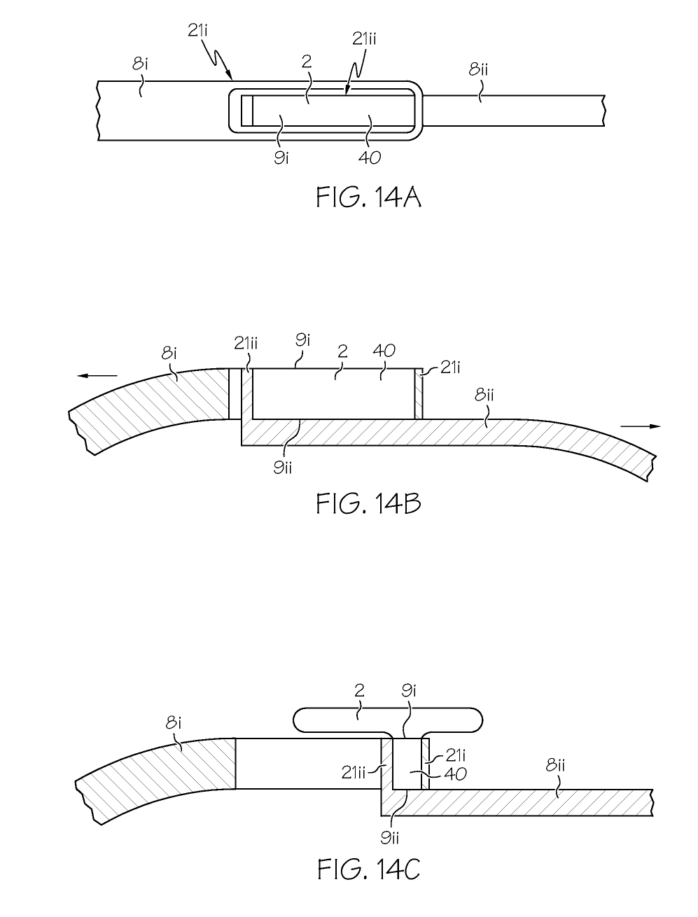

In at least one embodiment the reservoir can be positioned along a stent with interlocking members such as for example, the one used by REVA Medical Inc., of San Diego, Calif. an example of which is found in U.S. Pat. No. 6,951,053 which is hereby incorporated by reference in its entirety. As shown in overhead view FIG. 14A and side view FIG. 14B, the unexpanded stent comprises a first rib member (8i) and a second rib member (8ii) each slidably relative to the other and each ending with a tab (21i, 21ii), the two tabs (21i, 21ii) blocking any further sliding movement of the two ribs (8i, 8ii) when coming into contact with each other. The first rib (8i) has a slot (40) proportioned to fit the second rib's (8ii) tab (21ii) which guides the allowed movement of the second rib's (8ii) sliding movements. The slot (40) is a hollow open from the end (9ii) of the first rib facing the second rib to the opposite side of the first rib (9i). A quantity of agent (2) is positioned within the slot (40). As FIG. 14B shows, when the stent is expanded the tabs (21i, 21ii) move closer together which squeezes the agent (2) out. Because the flow of the agent (2) is blocked on one side (9ii) by the second rib (8ii), the flow is efficiently directed out the other side (9i) towards the body vessel. In at least one embodiment the second rib (8ii) has holes and the slot is sealed at the side (9i) opposite the second rib (8ii) causing the agent (2) to be squeezed out the side of the slot (40) where the second rib is located.

In some embodiments the stent, its delivery system, or other portion of an assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

This completes the description of the preferred and alternate embodiments of the invention. The above disclosure is intended to be illustrative and not exhaustive. This description will suggest many variations and alternatives to one of ordinary skill in this art. The various elements shown in the individual figures and described above may be combined, substituted, or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claims below.

1. An expandable tubular stent having an unexpanded state and an expanded state, the stent comprising:
a wall, the wall defining a plurality of cell openings therethrough, the wall constructed of a plurality of interconnected stent members, wherein adjacent stent members surround the cell openings, at least one of the stent members defining at least one reservoir, the at least one reservoir having at least one therapeutic agent releasably contained therein, in the unexpanded state the at least one reservoir having a first shape and in the expanded state a second shape different than the first shape, wherein the change in shape of the at least one reservoir initiates release of the at least one therapeutic agent from the at least one reservoir.

2. The stent of claim 1 wherein the wall is constructed out of struts, each strut is joined to one circumferentially adjacent strut at its proximal end by a curved turn and is joined to another oppositely circumferentially adjacent strut at its distal end by a curved turn, at least one reservoir is positioned along a focal point on at least one stent member, the focal point is a position on the stent member which becomes more deformed by expansion into the expanded state than adjacent portions of that stent member.

3. The stent of claim 2 wherein in the unexpanded state, the agent is frictionally engaged in the reservoir but which is released from the reservoir in the expanded state.

4. The stent of claim 2 wherein the reservoir further comprises at least two sidewalls having tops, a deformation of the sidewalls moves the sidewall tops together which squeezes the agent out of the reservoir.

5. The stent of claim 2 wherein the reservoir further comprises at least two sidewalls having tops, in the unexpanded state, the agent being held within the reservoir by frictional engagement, a deformation of the sidewalls moves the sidewall tops away from each other which breaks the frictional engagement thereby releasing the agent.

6. The stent of claim 2 wherein the reservoir has a tapered shape, narrower at positions closer to the turn and wider at positions farther away from the turn.

7. The stent of claim 2 wherein in the unexpanded state, the agent is held in place by a cap positioned over the agent which is frictionally engaged in place by the sidewalls of the reservoir, in the expanded state at least some of the sidewalls move away from each other thereby releasing the cap, the agent is released from the reservoir in response to the cap release.

8. The stent of claim 2 wherein the focal point is the apex of a concave bend formed in the expanded state.

9. The stent of claim 2 wherein the focal point is the apex of a convex bend formed in the expanded state.

10. The stent of claim 1 wherein a perimeter of at least one reservoir is constructed in one shape selected from the list consisting of: channel shaped, teardrop shaped, square shaped, circular shaped, oval shaped, polygonal shaped and any combination thereof.

11. The stent of claim 1 wherein the reservoir is a hole in the solid material of a stent member, the walls of the reservoir descending into the solid member material according to curved, tapered, triangular, square, trapezoidal shapes, and any combination thereof.

12. The stent of claim 11 wherein the reservoir is a hole that extends completely through the stent member material, in the unexpanded state the hole exposes the reservoir to contact with an inflation balloon the stent is crimped upon.

13. The stent of claim 5 wherein the agent is broken by the deformation.

14. The stent of claim 5 wherein the agent is a flexible material which becomes highly stretched by the deformation and releases through increased surface area contact.

15. The stent of claim 1 wherein in the unexpanded state, the agent is covered by a capping material, in the unexpanded state the capping material is impermeable and prevents release of the agent, the capping material breaks by becoming deformed in the expanded state.

16. The stent of claim 1 wherein the stent members include strut columns interconnected by connectors, a portion of at least one connector defines an opening in which a reservoir is positioned, the opening becoming deformed by the expansion of the stent.

17. The stent of claim 16 wherein the stent columns comprise generally linear struts, each strut is joined to one circumferentially adjacent strut at its proximal end by a curved turn and is joined to another oppositely circumferentially adjacent strut at its distal end by a curved turn, the connectors linking a turn on one strut column to a turn on an adjacent strut column, apices of the curve of the interconnected turns face each other.

18. The stent of claim 16 wherein the stent columns comprise generally linear struts, each strut is joined to one circumferentially adjacent strut at its proximal end by a curved turn member and is joined to another oppositely circumferentially adjacent strut at its distal end by a curved turn, the connectors link a turn on one strut column to a turn on an adjacent strut column, apices of the interconnected turn curves face away from each other.

19. The stent of claim 1 wherein in the unexpanded state the agent is positioned above a layer of material, in the expanded state the material breaks and pushes agent out of the reservoir.

20. A stent having an unexpanded state and an expanded state, the stent constructed of a plurality of interconnected stent members, the stent comprising one or more reservoirs of beneficial agent positioned on a stent member, in the unexpanded state the beneficial agent being retained within the one or more reservoir more securely than in the expanded state, the one or more reservoir comprising sidewalls,
at least one stent member comprises a focal point at a position on the member which becomes more deformed by expansion into the expanded state as sidewalls are moved closer together than adjacent portions of that member, the one or more reservoir being positioned on the focal point and the deformation causing a change in how securely the beneficial agent is retained within the reservoir.
